(19) United States
(12) Patent Application Publication
(10) Pub. No.: US 2009/0118809 A1
(43) Pub. Date: May 7, 2009

(54) ENDOPROSTHESIS WITH POROUS RESERVOIR AND NON-POLYMER DIFFUSION LAYER

(76) Inventors: Torsten Scheuermann, Munich (DE); Jan Weber, Maastricht (NL)

Correspondence Address: FISH & RICHARDSON PC, P.O. BOX 1022, MINNEAPOLIS, MN, 55440-1022, US

(21) Appl. No.: 11/934,342

(22) Filed: Nov. 2, 2007

(51) Int. Cl. A61F 2/82

(52) U.S. Cl. 623/1.4; 623/1.42; 623/1.39

(57) ABSTRACT

An endoprosthesis such as a coronary stent includes a porous reservoir of drug, e.g. directly in the body of the stent, and an overlayer formed of ceramic or metal for controlling elution of drug from the reservoir

### TECHNICAL FIELD

This disclosure relates to endoprostheses with a porous reservoir and non-polymer diffusion layer.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, e.g., so that it can contact the walls of the lumen. Stent delivery is further discussed in Heath, U.S. Pat. No. 6,290,721, the entire contents of which is hereby incorporated by reference herein.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

### SUMMARY

In an aspect, the invention features an endoprosthesis having a porous metal surface region, and a layer over the porous metal surface formed of porous ceramic or metal.

In another aspect, the invention features a method of forming an endoprosthesis that includes forming a porous metal surface on the endoprosthesis, introducing a drug into the porous metal surface, and forming a layer of porous ceramic or metal over the drug-containing porous metal surface.

Embodiments may also include one or more the following features. The porous metal surface region can include a drug. The layer can have a different porosity than the metal surface region. The layer can be less porous than the metal surface. The metal surface can have a plurality of cavities having a cross section of about 0.1 to 5 microns. The pore size of the layer can be smaller than the pore size of the metal surface. The pore size of the layer can be about 1-20 nm. The density of the drug can be about 0.5 μg/mm² or more. The thickness of the layer can be less than the thickness of the porous metal surface. The thickness of the layer can be about 10 to 500 nm. The thickness of the porous metal surface can be about 0.1 to 3 microns. The porous metal surface can be the surface of a stent body. The porous metal surface can be formed of stainless steel. The layer can be formed of metal. The layer can be formed of stainless steel. The porous metal surface and the layer can form a drug delivery system substantially free of polymer. The layer can be formed of ceramic. The ceramic can be IROX. The ceramic can have a striated morphology.

Embodiments may also include one or more the following features. The porous metal surface can be formed by ion bombardment. The metal surface can be formed on the body of a stent. The drug can be introduced by pulsed laser deposition (PLD). The layer can be formed by PLD. The layer can be a metal. The layer can be formed of the same metal as the porous metal surface. The layer can be ceramic.

Embodiments may include one or more of the following advantages. Stents can be formed with high loadings of drug on select portions, such as the abluminal surface, and the drug delivery profile can be carefully controlled using an over layer of a metal or a ceramic, without the use of a polymer. The drug can be loaded directly into the body of the stent, in porous regions in the stent surface metal. The porous region can have a high porosity, large pore openings, and large void cavities which can accommodate substantial amount of drug and can be relatively easily loaded by solvent techniques such as dipping or spraying, or direct dry loading of the drug into the porous region. The drug can be delivered to the porous region before the overlayer is provided, such that the drug can be delivered directly into the void regions without having to pass through the pores of the over layer. The over layer can be formed of a ceramic, e.g. IROX, which can have therapeutic advantages such as reducing the likelihood of restenosis and enhancing endothelialization. The morphology of the ceramic can be controlled to tune the therapeutic properties and the porosity of the over layer to provide a desired drug release profile over an extended period. The over layer can be a metal that is compatible with the porous surface region of the stent. For example, the over layer can be formed of the same metal as the stent porous region, which enhances bonding, biocompatibility, and reduces likelihood of degradation through corrosion. The porosity of the layer can be carefully controlled, e.g. the pore size can be controlled by laser drilling such that a desired drug elution profile results over a long period of time. The over layer can be formed by low temperature deposition process, such as PLD, which avoid degradation of drug previously provided in the porous region. The porous region can be highly porous for accommodating a large quantity of drug and at the same time relatively thin, so as not to degrade the performance of the stent. Likewise, the over layer can be relatively thin, so as not to substantially increase the overall thickness of the stent wall. A polymer carrier can be avoided, which reduces the likelihood of polymer delamination and facilitates deployment from a delivery device during deployment.

Still further aspects, features, embodiments, and advantages follow.

### DESCRIPTION OF DRAWINGS

FIGS. 1A-1C are longitudinal cross-sectional views illustrating delivery of a stent in a collapsed state, expansion of the stent, and deployment of the stent.

FIG. 2 is a perspective view of a stent.

FIGS. 3A-3C are cross-sectional views of a stent wall.

FIG. 4 is a cross-sectional schematic of drug elution.

FIG. 5 is a flow diagram illustrating manufacture of a stent.

FIGS. 6A-6C are schematics of an ion bombardment system.

FIG. 7 is a schematic of a PLD system.

FIGS. 8A and 8B are enlarged plan views of a stent wall surface.

FIGS. 9A-9C are schematic views of ceramic morphologies.

FIG. 10 is an SEM image of a porous surface.

### DETAILED DESCRIPTION

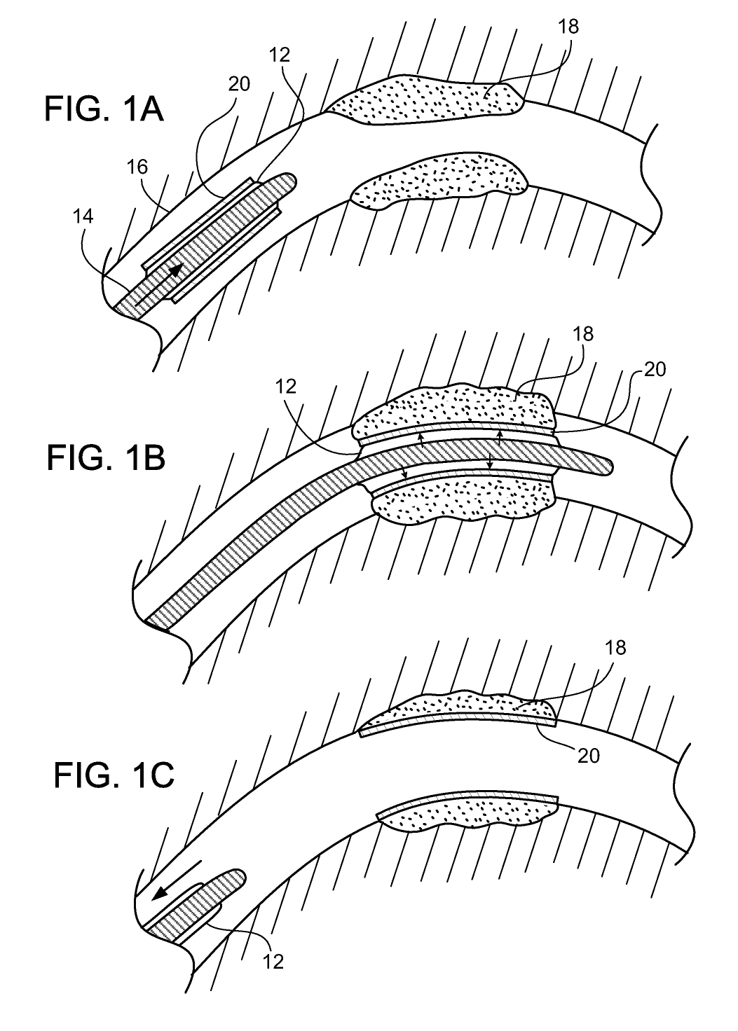

Referring to FIGS. 1A-1C, a stent 20 is placed over a balloon 12 carried near a distal end of a catheter 14, and is directed through the lumen 16 (FIG. 1A) until the portion carrying the balloon and stent reaches the region of an occlusion 18. The stent 20 is then radially expanded by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C).

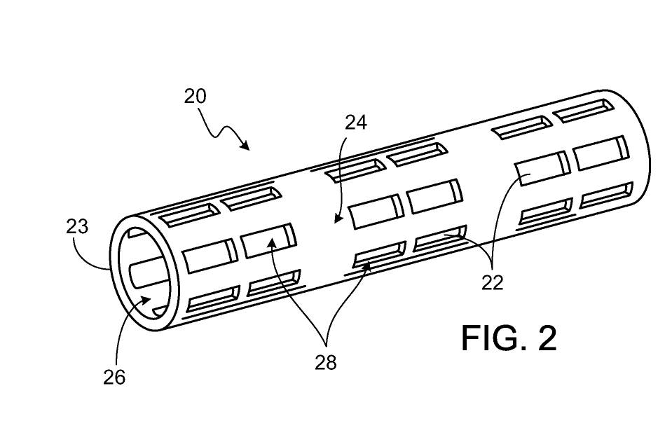

Referring to FIG. 2, the stent 20 includes a plurality of fenestrations 22 defined in a wall 23. Stent 20 includes several surface regions, including an outer, or abluminal, surface 24, an inner, adluminal, surface 26, and a plurality of cutface surfaces 28. The stent can be balloon expandable, as illustrated above, or a self-expanding stent. Examples of stents are described in Heath '721, supra.

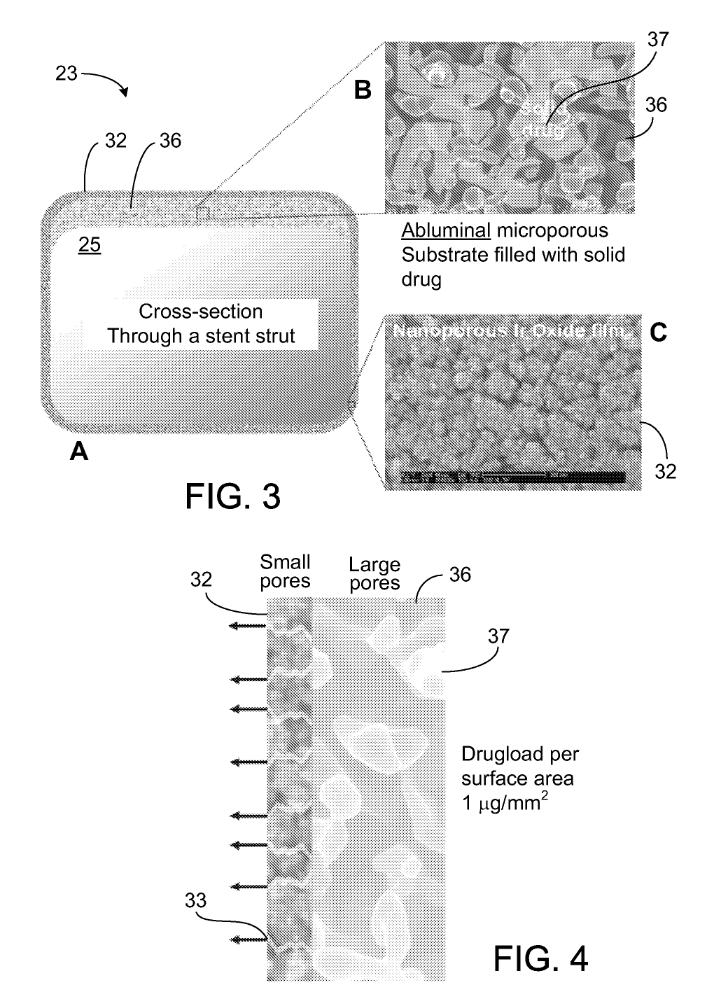

Referring to FIG. 3A, a cross-sectional view, a stent wall 23 includes a stent body 25 formed, e.g. of a metal, and includes a ceramic or metal layer 32 on the abluminal, adluminal, and cutface sides. The abluminal side also includes a porous region 36, which can be an integral surface portion of the sent body 25. Referring to FIG. 3B, the porous region has void regions in which a drug 37 is stored. Referring to FIG. 3C, the ceramic or metal layer 32 is also porous, but with generally smaller pores than the porous region. Referring as well to FIG. 4, the ceramic or metal layer 32 with small pores 33 modulates the diffusion of drug from the porous region 36 to provide a desired release profile.

The porous region can be formed with high porosity and large void regions which can accommodate large volumes of drug, without premature release of excessive doses of drug because the ceramic or metal layer modulates the drug release profile. Moreover, the high porosity and large void areas accommodate a substantial amount of drug, such that the porous region is relatively thin and thus does not substantially degrade the stent mechanical performance. In embodiments, the porous region is formed directly in the outer surface of a stent body, e.g. of stainless steel, without depositing a separate reservoir layer over the body. In particular embodiments, the porosity ratio (the ratio of the void volume to metal volume) is about 1:2, or more, e.g. about 1:1 or more, e.g. about 3:2. The drug loading per stent surface area (assuming a drug density of about 1 mg/mm³, the porous region thickness of about 3 μm, and 50% of the void regions filled with drug) is about 0.5 μg/mm² or more, e.g. about 1 μg/mm² or more, e.g. about 4 μg/mm². The void diameter is in the range of about 0.1 to 5 micron, e.g., about 0.5 to 3 micron. The thickness of the porous region is about five times the size of the pore diameter or less, e.g. about 0.3 to 15 microns, preferably about 0.5 to 5 micron. The ceramic or metal layer is selected for compatibility for the porous region and to have a controlled drug elution and therapeutic properties. In embodiments, the layer has a pore size of about 1 to 30 nm and a thickness of about 10 to 500 nm. In particular embodiments, the ceramic or metal overlayer has a gradually varying pore sizes through the thickness of the layer, e.g., relatively large pores close to the porous region and small pores close to the outmost surface of the layer. Such a configuration may allow better adherence of the overlayer to the porous region.

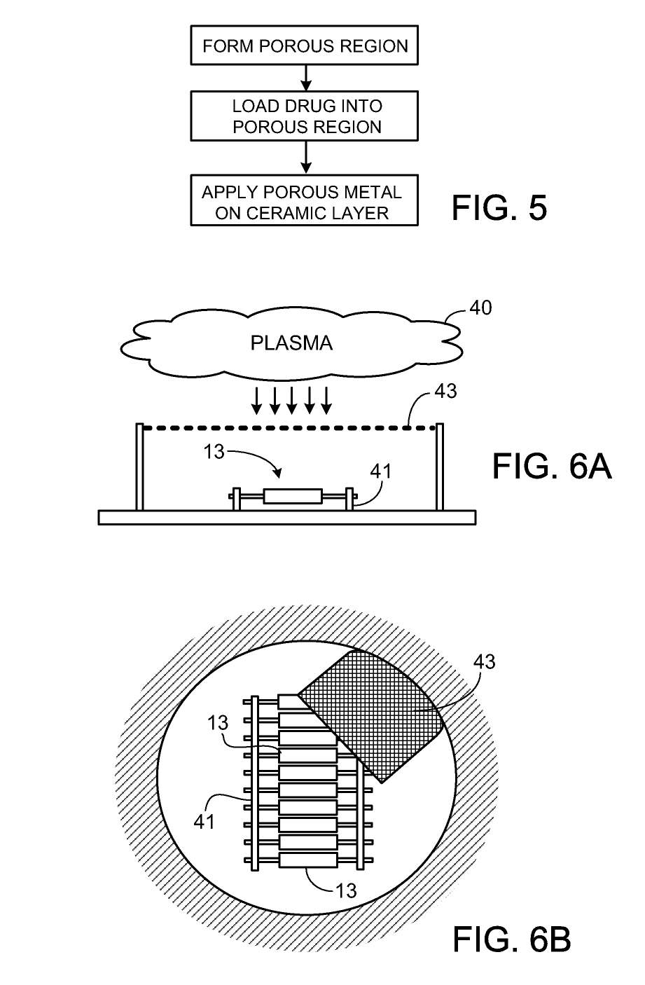

Referring to FIG. 5, the stent is formed by first providing the porous region on the stent. Next, a drug is delivered into the voids of the porous region. Finally, the ceramic or metal layer is provided over the porous layer by a technique that uses low temperature to avoid damaging the drug or the porous region, such as PLD.

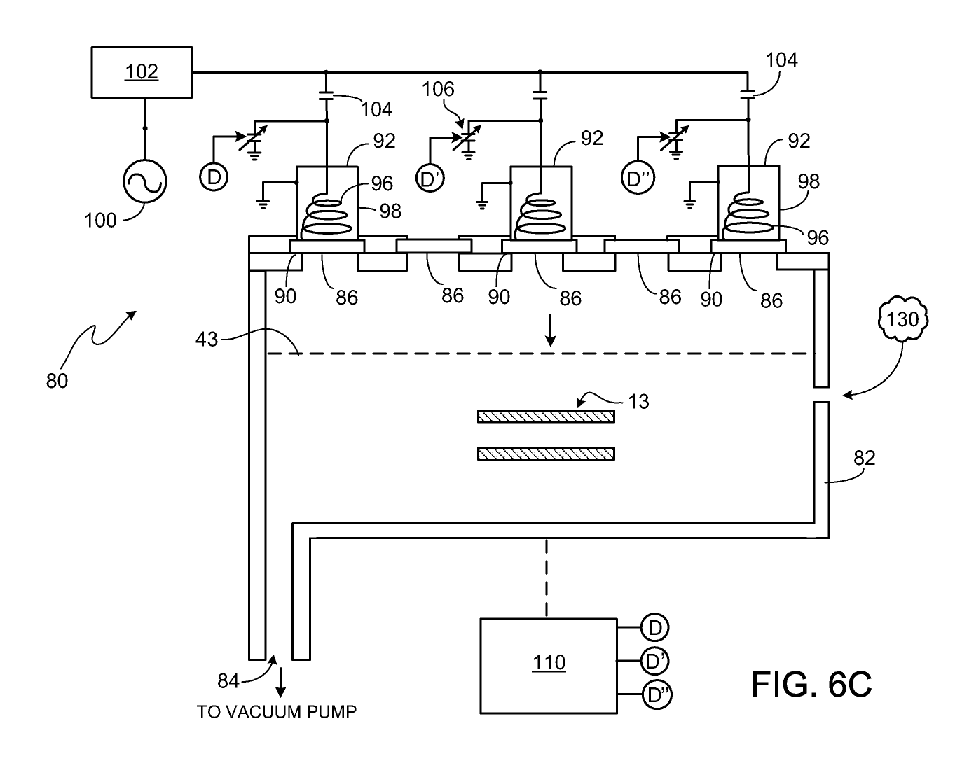

Referring to FIGS. 6A-6C, the porous surface can be formed, e.g., using an ion implantation process, such as plasma immersion ion implantation (“PIII”). Referring to FIGS. 6A and 6B, during PIII, charged species in a plasma 40, such as an Argon (or Krypton, or helium) plasma, are accelerated at high velocity towards stents 13, which are positioned on a sample holder 41. Acceleration of the charged species of the plasma towards the stents is driven by an electrical potential difference between the plasma and an electrode under the stent. Upon impact with a stent, the charged species, due to their high velocity, penetrate a distance into the stent and sputter the material of the stent, forming the porous regions discussed above. Generally, the porosity is controlled by controlling penetration depth, which is controlled, at least in part, by the potential difference between the plasma and the electrode under the stents. If desired, an additional electrode, e.g., in the form of a metal grid 43 positioned above the sample holder, can be utilized. Such a metal grid can be advantageous to prevent direct contact of the stents with the RF-plama between high-voltage pulses and can reduce charging effects of the stent material.

Referring to FIG. 6C an embodiment of a PIII processing system 80 includes a vacuum chamber 82 having a vacuum port 84 connected to a vacuum pump and a gas source 130 for delivering a gas, e.g., nitrogen, to chamber 82 to generate a plasma. System 80 includes a series of dielectric windows 86, e.g., made of glass or quartz, sealed by o-rings 90 to maintain a vacuum in chamber 82. Removably attached to some of the windows 86 are RF plasma sources 92, each source having a helical antenna 96 located within a grounded shield 98. The windows without attached RF plasma sources are usable, e.g., as viewing ports into chamber 82. Each antenna 96 electrically communicates with an RF generator 100 through a network 102 and a coupling capacitor 104. Each antenna 96 also electrically communicates with a tuning capacitor 106. Each tuning capacitor 106 is controlled by a signal D, D′, D″ from a controller 110. By adjusting each tuning capacitor 106, the output power from each RF antenna 96 can be adjusted to maintain homogeneity of the generated plasma. The regions of the stent directly exposed to ions from the plasma can be controlled by rotating the stents about their axis. The stents can be rotated continuously during treatment to enhance a homogenous modification of the entire stent. Alternatively, rotation can be intermittent, or selected regions can be masked, e.g., with a polymeric coating, to exclude treatment of those masked regions. A porous structure can be formed on only the abluminal surface by masking the inner stent lumen by mounting the stent on a metal rod. Pore size and cavity depth can be controlled by selecting the ion type, dosage per area, and substrate temperature, pulsing of the bombardment and kinetic energy. The substrate temperature is preferably 0.4 times or less of the melting temperature of the substrate temperature in Kelvin. The pulsing can be used to control substrate temperature to avoid overheating and weakening the metal substrate. For example, overheating can be avoided by using a pulse regime in which the continuous “ON” pulsing is replaced by several shorter “ON/OFF” cycles. The energy and dose of the incoming ions is significant enough to cause the substrate to heat without additional cooling or heat sink. However, when the dose is spread over time by pulsing one can compensate the incoming heat by sufficient cooling. Weakening of metals by excessive heating is a known effect. So-called sensitization is danger occurring when austenitic steel is heated in the range from 500° C. to 800° C. By this heating which occurs for example during welding the chrome in the stainless steel may react with the alloy's carbon forming chrome carbides. Although the overall temperature of the bombarded sample can be within range, the surface can be much higher in temperature. To avoid this effect the heat flux into the substrate (frequency of pulses in combination to density of plasma and voltage of pulses) is controlled such that it is smaller than the heat drain away from the surface. Heating is avoided by switching off the pulsation in intervals. The amount of heat input can be controlled by controlling parameters such as ion acceleration voltage (e.g. 20-35 kV), pulse frequency (e.g. 700 Hz), argon gas pressure (e.g. 0.2-0.4 Pa), RF power (e.g. 200 W), duty cycle of pulse generator (time on/(time off+time on)), pulse duration (in μs) (because the pulse shape (kV over μs) is not rectangular, everything that is below 10 kV is not effective and may be ignored), and arrangement of plasma source to substrate (e.g. geometry, distance). Further, the cycle time can be used with on time of 0.5 sec and an off time of 0.5 sec at a pulse voltage of 2 keV. Suitable plasma gases include nitrogen, argon, helium and xenon. In particular embodiments, for forming a porous surface on stainless steel, the plasma gas is argon, the ion energy is about 8-40 keV, and the ion dosage is about 1×10¹⁷ ions/cm². Additional details of PIII is described by Chu, U.S. Pat. No. 6,120,260; Brukner, Surface and Coatings Technology, 103-104, 227-230 (1998); Kutsenko, Acta Materialia, 52, 4329-4335 (2004); Guenzel, Surface & Coatings Technology, 136, 47-50, 2001; and Guenzel, J. Vacuum Science & Tech. B, 17(2), 895-899, 1999, the entire disclosure of each of which is hereby incorporated by reference herein. PIII is also discussed in U.S. Ser. No. 11/355,392, filed Feb. 16, 2006, and U.S. Ser. No. 11/355,368, filed Feb. 16, 2006.

A drug is loaded into the porous region. In embodiments, the drug is loaded prior to forming the ceramic or metal layer, which facilitates loading because the drug does not have to diffuse through the ceramic or metal layer to reach the porous region. In addition, the high porosity and large cavity size facilitate loading. In embodiments, the drug is loaded into porous region by dip coating or spraying the stent in a drug saturated solvent and drying under low temperature, e.g. ambient conditions. The drug is as a result precipitated into the porous region. The loading can be facilitated by repeatedly dipping and drying while the stent substrate is cooled under evacuated conditions. In embodiments, loading can also be facilitated by treating the porous region by corona discharge to make the surface more lipophilic, which attracts more lipophilic drugs to the surface. In embodiments, the drug is applied to the porous surface as a dry powder of small particles. The particles can be blown with a high velocity air jet deep into the porous surface. The surface can be treated by dip coating to further load the porous region. In embodiments, the drug particles are about 1 micron or less at their largest dimension, e.g. 500 nm or less. Suitable small particles, e.g. of paclitaxel, are available from Pharmasol GMBH, Blohmst 66 A, 12307 Berlin, Germany. In embodiments, the drug is applied to the porous region by a vapor deposition process, such as pulsed laser deposition. The drug can be deposited by providing drug as a target material in the PLD apparatus, as will be described further below. In embodiments, about 25% or more, e.g. about 50 to 90% of the void volume of the porous region is occupied by drug after loading. The surface of the porous region can be cleaned by exposure to a gas or fluid stream, e.g. flowed horizontally over the surface, to remove drug on the outermost regions so that the ceramic or metal layer is deposited directly onto the surfaces of the porous region to enhance layer adhesion and uniformity.

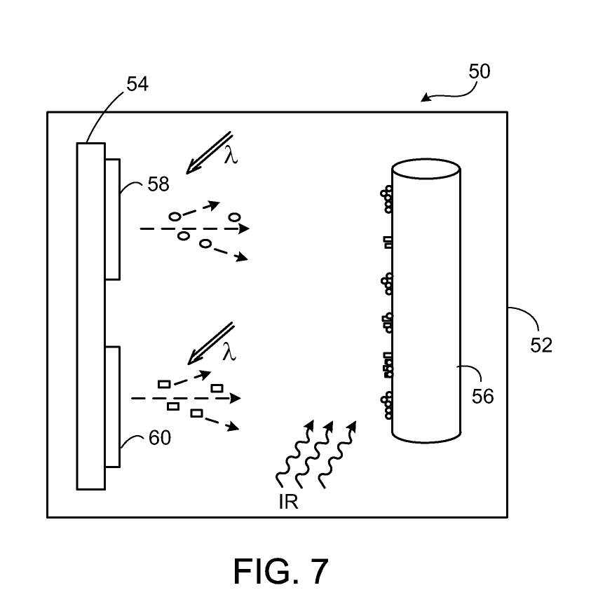

Referring to FIG. 7, in embodiments, the ceramic or metal layer is deposited by pulsed laser deposition (PLD). The PLD system 50 includes a chamber 52 in which is provided a target assembly 54 and a stent substrate 56, such as a stent body or a prestent structure such as a metal tube. The target assembly includes a first target material 58, such as a ceramic (e.g., IROX) or a precursor to a ceramic (e.g., iridium metal) or a metal, e.g. stainless steel and a second target material 60. Laser energy (double arrows) is selectively directed onto the target materials to cause the target materials to be ablated or sputtered from the target assembly. The sputtered material is imparted with kinetic energy in the ablation process such that the material is transported within the chamber (single arrows) and deposited on the stent 56. In addition, the temperature of the deposited material can be controlled by heating, e.g. using an infrared source (squiggly arrows).

The pore size of the ceramic film is controlled by varying the thickness, the laser power, the partial pressure of oxygen, the total pressure or the oxygen to argon ratio. In other embodiments, a PVD process is used by applying reactive sputtering from an iridium target under an oxygen atmosphere or an IROX target. In the case of a ceramic or a metal layer, the porosity can be further controlled by laser ablation of apertures into the layer with, e.g. a U.V. laser. As discussed above, the drug can also be applied to the porous layer by PLD. For example, the second target material 60 can be formed of drug. Laser energy applied to the second target material can sputter drug onto the porous surface, and/or can sputter drug with the ceramic or metal layer or sputter a layer of drug onto the ceramic or metal layer.

The porosity of the ceramic can be controlled by selecting the morphology, crystallinity, thickness, and size of the clusters ablated and deposited. Higher crystallinity, more defined grain morphologies, and thinner coatings provide greater porosity. Higher crystallinity and more defined grain morphologies can be formed by heating the deposited ceramic. Coating thickness is controlled by controlling deposition time. Higher laser energies can provide larger cluster sizes.

In particular embodiments, the laser energy is produced by an excimer laser operating in the ultraviolet, e.g. at a wavelength of about 248 nm (ArF), about 193 nm (ArF), or about 266 nm (Nd:YAG). The laser energy is about 100-700 mJ, the fluence is in the range of about 10 to 50 mJ/cm². The background pressure is in the range of about 1E-5 mbar to 1 mbar. The background gas includes oxygen. The substrate temperature is also controlled. The temperature of the substrate is between 25 to 300° C. during deposition. Substrate temperature can be controlled by directing an infrared beam onto the substrate during deposition using, e.g. a halogen source. The temperature is measured by mounting a heat sensor in the beam adjacent the substrate. The temperature can be varied to control the morphology of the ceramic material. The selective ablating of the ceramic or drug is controlled by mounting the target materials on a moving assembly that can alternately bring the materials into the path of the laser. Alternatively, a beam splitter and shutter can be used to alternatively or simultaneously expose multiple materials. PLD deposition services are available from Axyntec, Augsburg, Germany. Suitable ceramics include metal oxides and nitrides, such as of iridium, zirconium, titanium, hafnium, niobium, tantalum, ruthenium, platinum, and aluminum. In embodiments, the thickness of the coatings is in the range of about 50 μm to about 2 um, e.g. 100 nm to 500 nm. Pulsed laser deposition is also described in U.S. patent application Ser. No. 11/752,736, filed May 23, 2007 [Attorney Docket No. 10527-801001]. PLD is further described in Wang et al., Applied Surface Science 253: 2911-2914 (2006); Wang et al., Thin Solid Films 363: 58-60 (2000); and Zhang et al., Thin Solid Films 496: 371-375 (2006). Another suitable system is the Nano PLD system, from PVD Products, Inc., Wilmington, Mass. In embodiments, the laser is an ArF laser of 193 nm. For inorganic materials, a pulse laser energy density of about 2 J/cm² is used. For organic materials, such as SIBS agents, a pulse laser energy density of about 0.62 J/cm² to 0.9 J/cm₂ is used. In other embodiments, another physical vapor deposition (“PVD”) process is selected such as magnetron sputtering e.g. an iridium target under an oxygen atmosphere or an IROX target. Sputtering deposition is described in U.S. patent application Ser. No. 11/752,772, filed May 23, 2007 [Attorney Docket No. 10527-805001]. In the case of a ceramic or a metal over coating, the porosity can be further controlled by laser ablating apertures into the layer with, e.g. a U.V. laser.

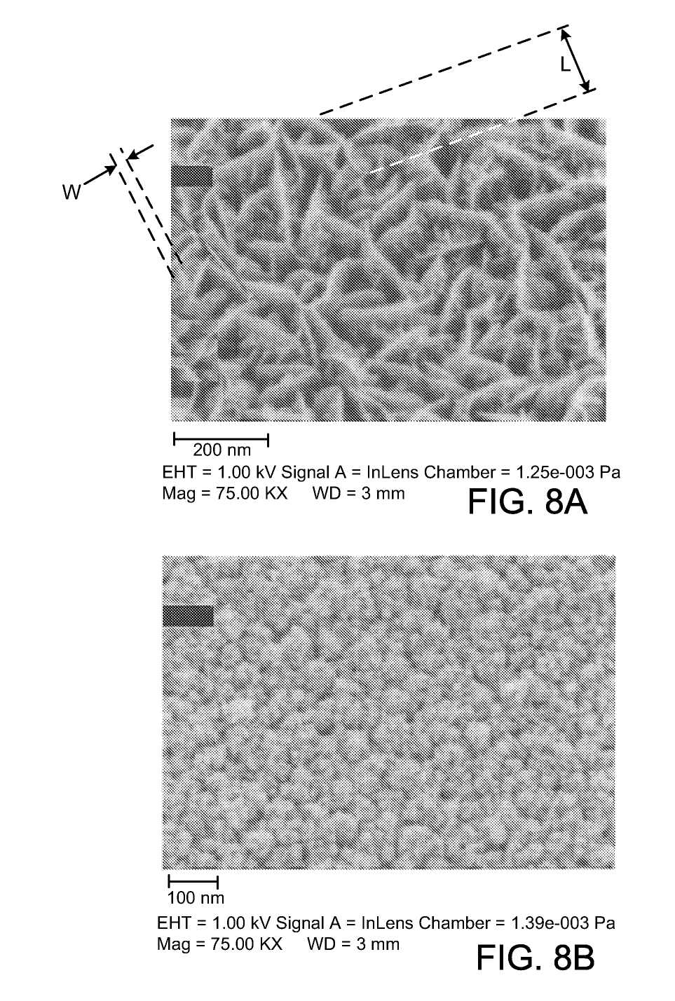

Referring to FIGS. 8A and 8B, the morphology of the ceramic can be varied between relatively rough surfaces and relatively smooth surfaces, which can each provide particular mechanical and therapeutic advantages, such as a controlled porosity to modulate drug release from the drug reservoir layer. Referring particularly to FIG. 8A, a ceramic coating can have a morphology characterized by defined grains and high roughness. Referring particularly to FIG. 8B, a ceramic coating can have a morphology characterized by a higher coverage, striated surface of generally lower roughness. The defined grain, high roughness morphology provides a high surface area characterized by crevices and generally higher porosity. Defined grain morphologies also allow for greater freedom of motion and are less likely to fracture as the stent is flexed in use and thus the coating resists delamination of the ceramic from an underlying. The stresses caused by flexure of the stent, during expansion or contraction of the stent or as the stent is delivered through a tortuously curved body lumen increase as a function of the distance from the stent axis. As a result, in embodiments, a morphology with defined grains is particularly desirable on abluminal regions of the stent or at other high stress points, such as the regions adjacent fenestrations which undergo greater flexure during expansion or contraction. Smoother globular surface morphology provides a surface which is tuned to facilitate endothelial growth by selection of its chemical composition and/or morphological features. Certain ceramics, e.g. oxides, can reduce restenosis through the catalytic reduction of hydrogen peroxide and other precursors to smooth muscle cell proliferation. The oxides can also encourage endothelial growth to enhance endothelialization of the stent. When a stent, is introduced into a biological environment (e.g., in vivo), one of the initial responses of the human body to the implantation of a stent, particularly into the blood vessels, is the activation of leukocytes, white blood cells which are one of the constituent elements of the circulating blood system. This activation causes a release of reactive oxygen compound production. One of the species released in this process is hydrogen peroxide, H₂O₂, which is released by neutrophil granulocytes, which constitute one of the many types of leukocytes. The presence of H₂O₂ may increase proliferation of smooth muscle cells and compromise endothelial cell function, stimulating the expression of surface binding proteins which enhance the attachment of more inflammatory cells. A ceramic, such as IROX can catalytically reduce H₂O₂. The smoother globular surface morphology of the ceramic can enhance the catalytic effect and reduce growth of smooth muscle cells.

The morphology of the ceramic is controlled by controlling the energy of the sputtered clusters on the stent substrate. Higher energies and higher temperatures result in defined grain, higher roughness surfaces. Higher energies are provided by increasing the temperature of the ceramic on the substrate, e.g. by heating the substrate or heating the ceramic with infrared radiation. In embodiments, defined grain morphologies are formed at temperatures of about 250° C. or greater. Globular morphologies are formed at lower temperatures, e.g. ambient temperatures without external factors. The heating enhances the formation of a more crystalline ceramic, which forms the grains. Intermediate morphologies are formed at intermediate values of these parameters. The composition of the ceramic can also be varied. For example, oxygen content can be increased by providing oxygen gas in the chamber.

The morphology of the surface of the ceramic is characterized by its visual appearance, its roughness, and/or the size and arrangement of particular morphological features such as local maxima. In embodiments, the surface is characterized by definable sub-micron sized grains. Referring particularly to FIG. 8A, for example, in embodiments, the grains have a length, L, of the of about 50 to 500 nm, e.g. about 100-300 nm, and a width, W, of about Sun to 50 nm, e.g. about 10-15 nm. The grains have an aspect ratio (length to width) of about 5:1 or more, e.g. 10:1 to 20:1. The grains overlap in one or more layers. The separation between grains can be about 1-50 nm. In particular embodiments, the grains resemble rice grains.

Referring particularly to FIG. 8B, in embodiments, the surface is characterized by a more continuous surface having a series of shallow globular features. The globular features are closely adjacent with a narrow minima between features. In embodiments, the surface resembles an orange peel. The diameter of the globular features is about 100 nm or less, and the depth of the minima, or the height of the maxima of the globular function is e.g. about 50 nm or less, e.g. about 20 nm or less. In other embodiments, the surface has characteristics between high aspect ratio definable grains and the more continuous globular surface and/or has a combination of these characteristics. For example, the morphology can include a substantially globular base layer and a relatively low density of defined grains. In other embodiments, the surface can include low aspect ratio, thin planar flakes. The morphology type is visible in FESEM images at 50 KX.

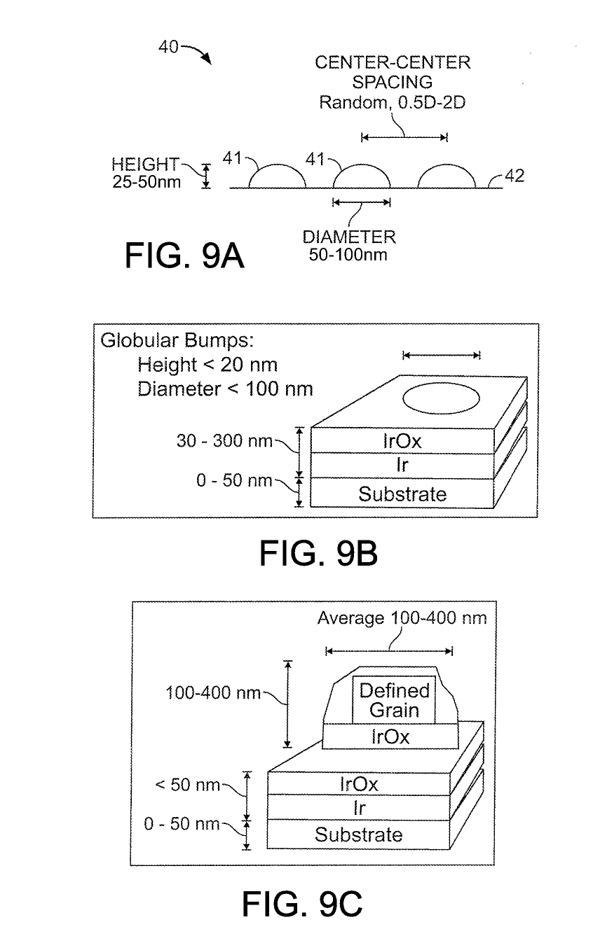

Referring to FIGS. 9A-9C, morphologies are also characterized by the size and arrangement of morphological features such as the spacing, height and width of local morphological maxima. Referring particularly to FIG. 9A, a coating 40 on a substrate 42 is characterized by the center-to-center distance and/or height, and/or diameter and/or density of local maxima. In particular embodiments, the average height, distance and diameter are in the range of about 400 nm or less, e.g. about 20-200 nm. In particular, the average center-to-center distance is about 0.5 to 2× the diameter.

Referring to FIG. 9B, in particular embodiments, the morphology type is a globular morphology, the width of local maxima is in the range of about 100 nm or less and the peak height is about 20 nm or less. In particular embodiments, the ceramic has a peak height of less than about 5 nm, e.g., about 1-5 nm, and/or a peak distance less than about 15 nm, e.g., about 10-15 nm. Referring to FIG. 9C, in embodiments, the morphology is defined as a grain type morphology. The width of local maxima is about 400 nm or less, e.g. about 100-400 nm, and the height of local maxima is about 400 nm or less, e.g. about 100-400 nm. As illustrated in FIGS. 9B and 9C, the select morphologies of the ceramic can be formed on a thin layer of substantially uniform, generally amorphous IROX, which is in turn formed on a layer of iridium metal, which is in turn deposited on a metal substrate, such as titanium or stainless steel. The spacing, height and width parameters can be calculated from AFM data.

The roughness of the surface is characterized by the average roughness, Sa, the root mean square roughness, Sq, and/or the developed interfacial area ratio, Sdr. The Sa and Sq parameters represent an overall measure of the texture of the surface. Sa and Sq are relatively insensitive in differentiating peaks, valleys and the spacing of the various texture features. Surfaces with different visual morphologies can have similar Sa and Sq values. For a surface type, the Sa and Sq parameters indicate significant deviations in the texture characteristics. Sdr is expressed as the percentage of additional surface area contributed by the texture as compared to an ideal plane the size of the measurement region. Sdr further differentiates surfaces of similar amplitudes and average roughness. Typically Sdr will increase with the spatial intricacy of the texture whether or not Sa changes.

In embodiments, the ceramic has a defined grain type morphology. The Sdr is about 30 or more, e.g. about 40 to 60. In addition or in the alternative, the morphology has an Sq of about 15 or more, e.g. about 20 to 30. In embodiments, the Sdr is about 100 or more and the Sq is about 15 or more. In other embodiments, the ceramic has a striated type surface morphology. The Sdr is about 20 or less, e.g. about 8 to 15. The Sq is about 15 or less, e.g. about less than 8 to 14. In still other embodiments, the ceramic has a morphology between the defined grain and the striated surface, and Sdr and Sq values between the ranges above, e.g. an Sdr of about 1 to 200 and/or an Sq of about 1 to 30.

The morphology of the ceramic coating can exhibit high uniformity. The uniformity provides predictable, tuned therapeutic and mechanical performance of the ceramic. The uniformity of the morphology as characterized by Sa, Sq or Sdr and/or average peak spacing parameters can be within about +/−20% or less, e.g. +/−10% or less within a 1 μm square. In a given stent region, the uniformity is within about +/−10%, e.g. about +/−1%. For example, in embodiments, the ceramic exhibits high uniformity over an entire surface region of stent, such as the entire abluminal or adluminal surface, or a portion of a surface region, such as the center 25% or 50% of the surface region. The uniformity is expressed as standard deviation. Uniformity in a region of a stent can be determined by determining the average in five randomly chosen 1 μm square regions and calculating the standard deviation. Uniformity of a morphology type in a region is determined by inspection of FESEM data at 50 kx.

The ceramics are also characterized by surface composition, composition as a function of depth, and crystallinity. In particular, the amounts of oxygen or nitride in the ceramic is selected for a desired catalytic effect on, e.g., the reduction of H₂O₂ in biological processes. The composition of metal oxide or nitride ceramics can be determined as a ratio of the oxide or nitride to the base metal. In particular embodiments, the ratio is about 2 to 1 or greater, e.g. about 3 to 1 or greater, indicating high oxygen content of the surface. In other embodiments, the ratio is about 1 to 1 or less, e.g. about 1 to 2 or less, indicating a relatively low oxygen composition. In particular embodiments, low oxygen content striated morphologies are formed to enhance endothelialization. In other embodiments, high oxygen content defined grain morphologies are formed, e.g., to enhance adhesion and catalytic reduction. Composition can be determined by x-ray photoelectron spectroscopy (XPS). Depth studies are conducted by XPS after FAB sputtering. The crystalline nature of the ceramic can be characterized by crystal shapes as viewed in FESEM images, or Miller indices as determined by x-ray diffraction. In embodiments, defined grain morphologies have a Miller index of <101>. Striated materials have blended amorphous and crystalline phases that vary with oxygen content. Higher oxygen content typically indicates greater crystallinity. Further discussion of ceramics and ceramic morphology and computation of roughness parameters is provided in U.S. patent application Ser. No. 11/752,736, filed May 23, 2007 [Attorney Docket No. 10527-801001], U.S. patent application Ser. No. 11/752,772, filed May 23, 2007 [Attorney Docket No. 10527-805001], and appendices.

In embodiments, ceramic is adhered only on the abluminal surface of the stent. This construction may be accomplished by, e.g. coating the stent before forming the fenestrations. In other embodiments, ceramic is adhered only on abluminal and cutface surfaces of the stent. This construction may be accomplished by, e.g., coating a stent containing a mandrel, which shields the luminal surfaces. Masks can be used to shield portions of the stent. In embodiments, the stent metal can be stainless steel, chrome, nickel, cobalt, tantalum, superelastic alloys such as nitinol, cobalt chromium, MP35N, and other metals. Suitable stent materials and stent designs are described in Heath '721, supra. In embodiments, the morphology and composition of the ceramic are selected to enhance adhesion to a particular metal. For example, in embodiments, the ceramic is deposited directly onto the metal surface of a stent body, e.g. a stainless steel, without the presence of an intermediate metal layer. As discussed above, different ceramic materials can be provided in different regions of a stent. For example, different materials may be provided on different stent surfaces. A rougher, defined grain material may be provided on the abluminal surface to, e.g. enhance adhesion while a striated material can be provided on the adluminal surface to enhance endothelialization. In embodiments, the drug is provided directly into the porous surface without a polymer. In other embodiments, the drug is applied to the porous surface with a polymer. Suitable polymers include, for example, copolymers thereof with vinyl monomers such as isobutylene, isoprene and butadiene, for example, styrene-isobutylene-styrene (SIBS), styrene-isoprene-styrene (SIS) copolymers, styrene-butadiene-styrene (SBS) copolymers. Other suitable polymers are discussed in U.S. patent application Ser. No. 11/752,736, filed May 23, 2007 [Attorney Docket No. 10527-801001]. The polymer is preferably capable of absorbing a substantial amount of drug solution. When applied as a coating on a medical device in accordance with the present invention, the dry polymer is typically on the order of from about 1 to about 50 microns thick, preferably about 1 to 10 microns thick, and more preferably about 2 to 5 microns. Very thin polymer coatings, e.g., of about 0.2-0.3 microns and much thicker coatings, e.g., more than 10 microns, are also possible. Multiple layers of polymer coating can be provided onto a medical device. Such multiple layers are of the same or different polymer materials.

### EXAMPLE

A stainless steel surface is treated by PIII bombardment to form a porous surface. The treatment is carried out at the large chamber at Rossendorf Research Center (Geunzel, Surface & Coating Technology, 136, 47-50, 2001 and J. Vacuum Science & Techn. B, 17(2), 895-899, 1999). The operating conditions are given in the Table below.

TABLE

| | |
|---|---|
| Ion type: | argon |
| Ion energy: | 35 keV |
| Ion dose: | 20 × 10¹⁷ ions/cm² |
| RF frequency: | 800 Hz |
| Pulse duration: | 5 μs |
| Power of radio frequency pulse: | 350 W |
| Argon pressure: | 0.2 Pa |
| Substrate temperature | 420° C. |

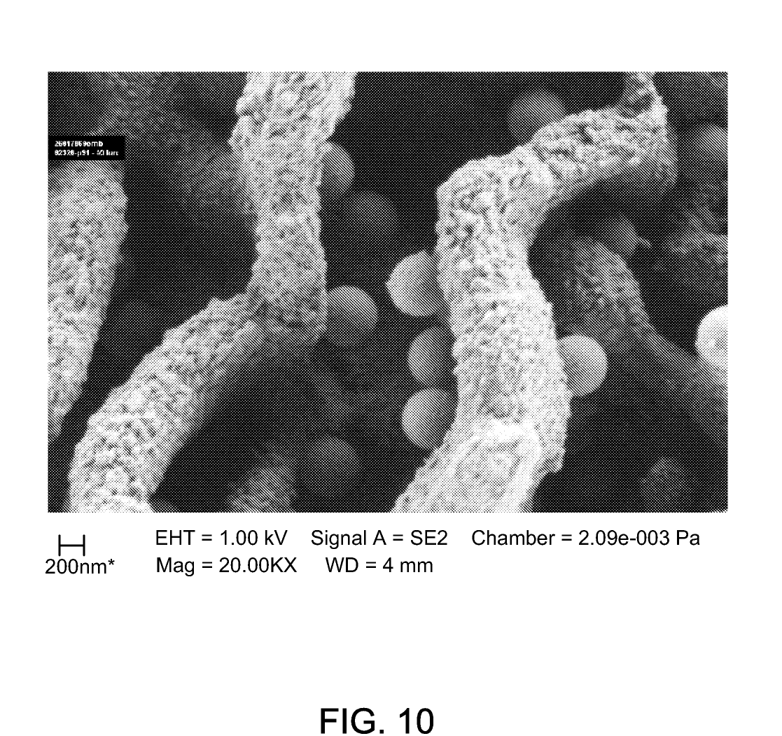

Referring to FIG. 10, an SEM image of the surface, a highly porous structure is formed having surface openings greater than a micron and about 2.5 microns deep. The spheres in the image are formed of polystyrene covered with a layer of silica and have a diameter of about 500 nm.

The terms “therapeutic agent”, “pharmaceutically active agent”, “pharmaceutically active material”, “pharmaceutically active ingredient”, “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total.

Exemplary therapeutic agents include, e.g., anti-thrombogenic agents (e.g., heparin); anti-proliferative/anti-mitotic agents (e.g., paclitaxel, 5-fluorouracil, cisplatin, vinblastine, vincristine, inhibitors of smooth muscle cell proliferation (e.g., monoclonal antibodies), and thymidine kinase inhibitors); antioxidants; anti-inflammatory agents (e.g., dexamethasone, prednisolone, corticosterone); anesthetic agents (e.g., lidocaine, bupivacaine and ropivacaine); anti-coagulants; antibiotics (e.g., erythromycin, triclosan, cephalosporins, and aminoglycosides); agents that stimulate endothelial cell growth and/or attachment. Therapeutic agents can be nonionic, or they can be anionic and/or cationic in nature. Therapeutic agents can be used singularly, or in combination. Preferred therapeutic agents include inhibitors of restenosis (e.g., paclitaxel), anti-proliferative agents (e.g., cisplatin), and antibiotics (e.g., erythromycin). Additional examples of therapeutic agents are described in U.S. Published Patent Application No. 2005/0216074. Polymers for drug elution coatings are also disclosed in U.S. Published Patent Application No. 2005/019265A. A functional molecule, e.g. an organic, drug, polymer, protein, DNA, and similar material can be incorporated into groves, pits, void spaces, and other features of the ceramic.

The stents described herein can be configured for vascular, e.g. coronary and peripheral vasculature or non-vascular lumens. For example, they can be configured for use in the esophagus or the prostate. Other lumens include biliary lumens, hepatic lumens, pancreatic lumens, uretheral lumens and uretheral lumens.

Any stent described herein can be dyed or rendered radiopaque by addition of, e.g., radiopaque materials such as barium sulfate, platinum or gold, or by coating with a radiopaque material. The stent can include (e.g., be manufactured from) metallic materials, such as stainless steel (e.g., 316L, BioDur® 108 (UNS S29108), and 304L stainless steel, and an alloy including stainless steel and 5-60% by weight of one or more radiopaque elements (e.g., Pt, Ir, Au, W) (PERSS®) as described in US-2003-0018380-A1, US-2002-0144757-A1, and US-2003-0077200-A1), Nitinol (a nickel-titanium alloy), cobalt alloys such as Elgiloy, L605 alloys, MP35N, titanium, titanium alloys (e.g., Ti-6A1-4V, Ti-50Ta, Ti-10Ir), platinum, platinum alloys, niobium, niobium alloys (e.g., Nb-1Zr) Co-28Cr-6Mo, tantalum, and tantalum alloys. Other examples of materials are described in commonly assigned U.S. application Ser. No. 10/672,891, filed Sep. 26, 2003; and U.S. application Ser. No. 11/035,316, filed Jan. 3, 2005. Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; and commonly assigned U.S. application Ser. No. 10/346,487, filed Jan. 17, 2003.

The stent can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., U.S. Pat. No. 6,290,721). The ceramics can be used with other endoprostheses or medical devices, such as catheters, guide wires, and filters.

All publications, patent applications, and patents, are incorporated by reference herein in their entirety.

Still other embodiments are in the following claims.

What is claimed is:

1. An endoprosthesis, comprising:
a porous metal surface, and
a layer over the porous metal surface region formed of porous ceramic or metal.

2. The endoprosthesis of claim 1 wherein the porous metal surface region includes a drug.

3. The endoprosthesis of claim 2 wherein the layer has different porosity than the metal surface region.

4. The endoprosthesis of claim 3 wherein the layer is less porous than the metal surface.

5. The endoprosthesis of claim 1 wherein the metal surface has a plurality of cavities having a cross section of about 0.1 to 5 microns.

6. The endoprosthesis of claim 4 wherein the pore size of the layer is smaller than the pore size of the metal surface.

7. The endoprosthesis of claim 4 wherein the pore size of the layer is about 1 to 20 nm.

8. The endoprosthesis of claim 2 wherein the density of the drug is about 0.5 μg/mm² or more.

9. The endoprosthesis of claim 1 wherein the thickness of the layer is less than the thickness of the porous metal surface.

10. The endoprosthesis of claim 9 wherein the thickness of the layer is about 10 to 500 nm.

11. The endoprosthesis of claim 9 wherein the thickness of the porous metal surface is about 0.1 to 3 microns.

12. The endoprosthesis of claim 1 wherein the porous metal surface is the surface of a stent body.

13. The endoprosthesis of claim 1 wherein the porous metal surface is formed of stainless steel.

14. The endoprosthesis of claim 13 wherein the layer is formed of metal.

15. The endoprosthesis of claim 13 wherein the layer is formed of stainless steel.

16. The endoprosthesis of claim 1 wherein the porous metal surface and the layer form a drug delivery system substantially free of polymer.

17. The endoprosthesis of claim 1 wherein the layer is formed of ceramic.

18. The endoprosthesis of claim 17 wherein the ceramic is IROX.

19. The endoprosthesis of claim 17 wherein the ceramic has a striated morphology.

20. A method of forming an endoprosthesis, comprising:
forming a porous metal surface on the endoprosthesis,
introducing a drug into the porous metal surface, and
forming a layer of porous ceramic or metal over the drug-containing porous metal surface.

21. The method of claim 20 comprising forming the porous metal surface by ion bombardment.

22. The method of claim 21 wherein the metal surface is on the body of a stent.

23. The method of claim 20 comprising introducing the drug by PLD.

24. The method of claim 20 comprising forming the layer by PLD.

25. The method of claim 20 wherein the layer is a metal.

26. The method of claim 25 wherein the layer is formed of the same metal as the porous metal surface.

27. The method of claim 20 wherein the layer is ceramic.
